(19) United States
(12) Patent Application Publication
(10) Pub. No.: US 2007/0202052 A1
(43) Pub. Date: Aug. 30, 2007

(54) Method for increasing the expression of pulmonary surfactant protein-B

(76) Inventors: Thomas Brenna, Ithaca, NY (US); Kumar Sesha Durga Kothapalli, Ithaca, NY (US); Zeina Jouni, Evansville, IN (US); Joshua C. Anthony, Evansville, IN (US); Steven C. Rumsey, Curitiba (BR)

Correspondence Address: Richard D. Schmidt;Bristol-Myers Squibb Company, 2400 West Lloyd Expressway, Evansville, IN, 47721-0001, US

(21) Appl. No.: 11/711,197

(22) Filed: Feb. 27, 2007

Related U.S. Application Data
(60) Provisional application No. 60/777,344, filed on Feb. 28, 2006.

(51) Int. Cl. A61K 31/202; A61K 9/12

(52) U.S. Cl. 424/45; 514/560

(57) ABSTRACT

The present invention is directed to a novel method for increasing the expression of pulmonary surfactant protein-B in an infant. The method comprises administration of a therapeutically effective amount of DHA and ARA, alone or in combination with one another, to the infant.

This application claims the priority benefit of U.S. Provisional Application 60/777,344 filed Feb. 28, 2006 which is incorporated by reference herein in its entirety.

### BACKGROUND OF THE INVENTION

(1) Field of the Invention

The present invention relates generally to a method for inducing the expression of pulmonary surfactant protein-B.

(2) Description of the Related Art

If an infant is to breathe properly upon birth, the small air sacs (alveoli) at the ends of the breathing tubes in the lungs must open with the first breath and remain open during the breathing cycle so that oxygen in the air can be absorbed into the blood vessels that surround the alveoli. The walls of the alveoli are coated with a thin film of water, posing a potential problem in keeping them open. Surface tension is created inside the small alveoli because the water molecules are more attracted to each other than to air. As the infant exhales and the alveoli contract, the water molecules come closer together and the surface tension increases. Potentially, without a countering mechanism in the body, the increased surface tension could cause the alveoli to collapse and would make it extremely difficult to re-expand the alveoli upon inhalation.

Pulmonary surfactant is a barrier material that naturally forms a layer between the alveolar surface and the alveolar gas, reducing the surface tension inside the alveoli. It allows the alveoli to expand with an infant's first breath and remain open throughout the normal cycle of inhalation and exhalation. Without an adequate supply of pulmonary surfactant, the alveoli may never inflate properly or may collapse upon exhalation and require an inordinate amount of force to re-expand on inhalation.

Pulmonary surfactant is a mixture of about 90% lipid and about 10% protein, synthesized and secreted into the alveolar fluid by the alveolar type II epithelial cells. The protein portion of pulmonary surfactant is comprised of four surfactant-specific proteins, designated as surfactant protein-A (SP-A), SP-B, SP-C, and SP-D. The hydrophilic surfactant proteins SP-A and SP-D are members of a family of collagenous carbohydrate-binding proteins, known as collecting. SP-A and SP-D are believed to be molecules of the innate immune system due to their ability to recognize a broad spectrum of pathogens.

SP-B and SP-C are hydrophobic membrane proteins that increase the rate at which surfactant spreads over the surface of alveoli. SP-B has been identified as an essential constituent of pulmonary surfactant and is required for proper biophysical function of the lung. The critical role of SP-B in lung function was first recognized in the study of an infant who died from respiratory failure in the postnatal period. The infant's death was found to be associated with a lack of SP-B protein or SP-B mRNA in airway secretions or lung tissue. Nogee, L. M., et al., Deficiency of Pulmonary Surfactant Protein B in Congenital Alveolar Proteinosis, N. Engl. J. Med. 328:406-410 (1993). A later study confirmed the importance of SP-B from observations that an inherited deficiency of SP-B causes mice to develop lethal respiratory disease. Nogee, L. M., et al., A Mutation in the Surfactant Protein B Gene Responsible for Fatal Neonatal Respiratory Disease in Multiple Kindreds, J. Clin. Invest. 93:1860-1863 (1994). Thus, it is generally recognized that SP-B plays a vital role in the function of pulmonary surfactant and respiratory health.

In humans, pulmonary surfactant is formed relatively late in fetal life, between about the 24th and 28th week of gestation. By about 35 weeks gestation, adequate amounts of surfactant have developed. An infant born prematurely, however, may not have adequate amounts of surfactant present in the lungs. In addition to prematurity, genetic predispositions or inherited disorders can cause a term infant to lack adequate supplies of surfactant. An infant born without an adequate supply of surfactant is likely to develop respiratory distress syndrome (RDS) immediately after birth.

RDS, also known as hyaline membrane disease, affects approximately 10% of all premature infants. Approximately half of all infants born between 28 and 32 weeks gestational age develop RDS. In RDS, the alveoli collapse due to a lack of surfactant, thereby preventing the infant from breathing properly. Symptoms usually appear shortly after birth and become progressively more severe. Symptoms can include rapid, short or unusual breathing, nasal flaring, a bluish skin color, swollen arms or legs, tachypnea, expiratory grunting due to a partial closure of the glottis, subcostal and intercostals retractions, cyanosis, apnea or hypothermia.

RDS can be diagnosed by blood gas analysis or a chest x-ray. Blood cultures and a sepsis work-up are usually conducted to rule out infection or sepsis as a cause of the respiratory distress. Once diagnosed, the infant is given high oxygen and humidity concentrations and may be placed on a ventilator. A biologic, animal-modified, or synthetic lung surfactant may be delivered into the lungs through an endotracheal tube. Although the incidence and severity of complications of RDS are reduced via these techniques, RDS continues to present significant infant morbidities.

Therefore, it would be beneficial to provide a composition that can induce the expression of pulmonary surfactant protein-B in infants and thereby prevent or treat RDS. It would be beneficial to provide a composition that allows infants to produce adequate supplies of their own pulmonary surfactant, alleviating the need for the administration of ventilation techniques or artificial surfactant. In addition, it would be beneficial to provide an infant formula containing such a composition in order to induce the expression of pulmonary surfactant protein-B in infants and prevent or treat RDS in infants.

### SUMMARY OF THE INVENTION

Briefly, the present invention is directed to a novel method for inducing the expression of pulmonary surfactant protein-B in a subject, the method comprising administering to the subject a therapeutically effective amount of DHA or ARA, alone or in combination with one another. The subject may be an infant or a child. In some embodiments, the ratio of ARA:DHA by weight may be about 1:1.5. In other embodiments, DHA comprises between about 0.33% and 1.00% of fatty acids by weight.

Among the several advantages found to be achieved by the present invention, it can prevent or treat respiratory distress syndrome in infants or children.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

As used herein, the term “inducing” means causing, bringing about or stimulating the occurrence of.

The terms “therapeutically effective amount” refer to an amount that results in an improvement or remediation of the disease, disorder, or symptoms of the disease or condition.

The term “infant” means a postnatal human that is less than about 1 year of age.

The term “child” means a human that is between about 1 year and 12 years of age. In some embodiments, a child is between the ages of about 1 and 6 years. In other embodiments, a child is between the ages of about 7 and 12 years.

As used herein, the term “infant formula” means a composition that satisfies the nutrient requirements of an infant by being a substitute for human milk. In the United States, the contents of an infant formula are dictated by the federal regulations set forth at 21 C.F.R. Sections 100, 106, and 107. These regulations define macronutrient, vitamin, mineral, and other ingredient levels in an effort to stimulate the nutritional and other properties of human breast milk.

In accordance with the present invention, the inventors have discovered a novel method for inducing the expression of pulmonary surfactant protein-B in a subject which comprises administering a therapeutically effective amount of docosahexaenoic acid (DHA) and arachidonic acid (ARA) to the subject. In fact, it has been shown in the present invention that the administration of 1.00% DHA and 0.67% ARA, as a percentage of total fafty acids, induces the expression of pulmonary surfactant protein-B by about 35% when compared to an unsupplemented group.

DHA and ARA are long chain polyunsaturated fatty acids (LCPUFA) which have been shown to contribute to the health and growth of infants. Specifically, DHA and ARA have been shown to support the development and maintenance of the brain, eyes and nerves of infants. Birch, E., et al., A Randomized Controlled Trial of Long-Chain Polyunsaturated Fatty Acid Supplementation of Formula in Term Infants after Weaning at 6 Weeks of Age, Am. J. Clin. Nutr. 75:570-580 (2002). Clandinin, M., et al., Formulas with Docosahexaenoic Acid (DHA) and Arachidonic Acid (ARA) Promote Better Growth and Development Scores in Very-Low-Birth-Weight Infants (VLBW), Pediatr. Res. 51:187A-188A (2002). DHA and ARA are typically obtained through breast milk in infants that are breast-fed. In infants that are formula-fed, however, DHA and ARA must be supplemented into the diet.

While it has been shown that DHA and ARA are beneficial to the development of brain, eyes and nerves in infants, neither DHA alone nor in combination with ARA has previously been shown to have any effect on the levels of pulmonary surfactant protein-B within the lungs. The positive effects of DHA alone and in combination with ARA on pulmonary surfactant protein-B that were discovered in the present invention were surprising and unexpected.

In some embodiments of the present invention, the subject is in need of the expression of pulmonary surfactant protein-B. The subject can have low levels of pulmonary surfactant protein-B in the lungs at birth, or the levels of pulmonary surfactant protein-B may decrease over time. Additionally, the subject in need of enhanced pulmonary surfactant protein-B levels may be at risk for developing respiratory distress syndrome. The subject can be at risk due to genetic predisposition, gestational age at birth, lung underdevelopment, multiple births, emergency caesarian section birth, diseases, disorders, and the like. For example, an infant born at less than 28 weeks gestational age is at risk for developing respiratory distress syndrome. As such, in certain embodiments the infant in need of the expression of pulmonary surfactant protein-B may be a preterm infant. As another example, a term infant born to a mother having chorioamnionitis or diabetes is at risk for developing respiratory distress syndrome and may be in need of the expression of pulmonary surfactant protein-B.

In the present invention, the form of administration of DHA or ARA, alone or in combination with one another, is not critical, as long as a therapeutically effective amount is administered to the subject. In some embodiments, the DHA or ARA, alone or in combination with one another, are administered to a subject via tablets, pills, encapsulations, caplets, gelcaps, capsules, oil drops, or sachets. In another embodiment, the DHA or ARA, alone or in combination with one another, are added to a food or drink product and consumed. The food or drink product may be a children's nutritional product such as a follow-on formula, growing up milk, or a milk powder or the product may be an infant's nutritional product, such as an infant formula.

In an embodiment, the infant formula for use in the present invention is nutritionally complete and contains suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, amino acids, and the like. Carbohydrate sources can be any used in the art, e.g., lactose, glucose, corn syrup solids, maltodextrins, sucrose, starch, rice syrup solids, and the like. Lipid sources can be any used in the art, e.g., vegetable oils such as palm oil, canola oil, corn oil, soybean oil, palmolein, coconut oil, medium chain triglyceride oil, high oleic sunflower oil, high oleic safflower oil, and the like.

Conveniently, commercially available infant formula can be used. For example, Enfalac, Enfamil®, Enfamil® Premature Formula, Enfamil® with Iron, Lactofree®, Nutramigen®, Pregestimil®, and ProSobee® (available from Mead Johnson & Company, Evansville, Ind., U.S.A.) may be supplemented with suitable levels of DHA or ARA, alone or in combination with one another, and used in practice of the method of the invention. Additionally, Enfamil® LIPIL®, which contains effective levels of DHA and ARA, is commercially available and may be utilized in the present invention.

The method of the invention requires the administration of a DHA or ARA, alone or in combination with one another. In this embodiment, the weight ratio of ARA:DHA is typically from about 1:3 to about 9:1. In one embodiment of the present invention, this ratio is from about 1:2 to about 4:1. In yet another embodiment, the ratio is from about 2:3 to about 2:1. In one particular embodiment the ratio is about 2:1. In another particular embodiment of the invention, the ratio is about 1:1.5. In other embodiments, the ratio is about 1:1.3. In still other embodiments, the ratio is about 1:1.9. In a particular embodiment, the ratio is about 1.5:1. In a further embodiment, the ratio is about 1.47:1.

In certain embodiments of the invention, the level of DHA is between about 0.0% and 1.00% of fatty acids, by weight. Thus, in certain embodiments, the ARA alone may treat or reduce obesity.

The level of DHA may be about 0.32% by weight. In some embodiments, the level of DHA may be about 0.33% by weight. In another embodiment, the level of DHA may be about 0.64% by weight. In another embodiment, the level of DHA may be about 0.67% by weight. In yet another embodiment, the level of DHA may be about 0.96% by weight. In a further embodiment, the level of DHA may be about 1.00% by weight.

In embodiments of the invention, the level of ARA is between 0.0% and 0.67% of fatty acids, by weight. Thus, in certain embodiments of the invention, DHA alone can treat or reduce obesity. In another embodiment, the level of ARA may be about 0.67% by weight. In another embodiment, the level of ARA may be about 0.5% by weight. In yet another embodiment, the level of DHA may be between about 0.47% and 0.48% by weight.

The effective amount of DHA in an embodiment of the present invention is typically from about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 15 mg per kg of body weight per day to about 60 mg per kg of body weight per day.

The effective amount of ARA in an embodiment of the present invention is typically from about 5 mg per kg of body weight per day to about 150 mg per kg of body weight per day. In one embodiment of this invention, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day.

The amount of DHA in infant formulas for use in the present invention typically varies from about 2 mg/100 kilocalories (kcal) to about 100 mg/100 kcal. In another embodiment, the amount of DHA varies from about 5 mg/100 kcal to about 75 mg/100 kcal. In yet another embodiment, the amount of DHA varies from about 15 mg/100 kcal to about 60 mg/100 kcal.

The amount of ARA in infant formulas for use in the present invention typically varies from about 4 mg/100 kilocalories (kcal) to about 100 mg/100 kcal. In another embodiment, the amount of ARA varies from about 10 mg/100 kcal to about 67 mg/100 kcal. In yet another embodiment, the amount of ARA varies from about 20 mg/100 kcal to about 50 mg/100 kcal. In a particular embodiment, the amount of ARA varies from about 25 mg/100 kcal to about 40 mg/100 kcal. In a further embodiment, the amount of ARA is about 30 mg/100 kcal.

The infant formula supplemented with oils containing DHA or ARA, alone or in combination with one another, for use in the present invention can be made using standard techniques known in the art. For example, replacing an equivalent amount of an oil normally present, e.g., high oleic sunflower oil.

The source of the ARA and DHA can be any source known in the art such as marine oil, fish oil, single cell oil, egg yolk lipid, brain lipid, and the like. The DHA and ARA can be in natural form, provided that the remainder of the LCPUFA source does not result in any substantial deleterious effect on the infant. Alternatively, the DHA and ARA can be used in refined form.

In one embodiment, the LCPUFA source contains eicosapentaenoic acid (EPA). In another embodiment, the LCPUFA source is substantially free of EPA. For example, the infant formulas used herein may contain less than about 20 mg/100 kcal EPA; in another embodiment less than about 10 mg/100 kcal EPA; in yet another embodiment less than about 5 mg/100 kcal EPA; and in a further embodiment substantially no EPA.

Sources of DHA and ARA may be single cell oils as taught in U.S. Pat. Nos. 5,374,657, 5,550,156, and 5,397,591, the disclosures of which are incorporated herein by reference in their entirety.

In an embodiment of the present invention, DHA or ARA, alone or in combination with one another, are supplemented into the diet of an infant from birth until the infant reaches about one year of age. In a particular embodiment, the infant can be a preterm infant. In another embodiment of the invention, DHA or ARA, alone or in combination with one another, are supplemented into the diet of a subject from birth until the subject reaches about two years of age. In other embodiments, DHA or ARA, alone or in combination with one another, are supplemented into the diet of a subject for the lifetime of the subject. Thus, in particular embodiments, the subject may be a child, adolescent, or adult. In still other embodiments, the DHA or ARA, alone or in combination with one another, are administered prenatally to the infant's mother. Prenatal administration of DHA or ARA, alone or in combination with one another, may induce the expression of SP-B in the unborn infant.

In an embodiment, the subject of the invention is a child between the ages of one and six years old. In another embodiment the subject of the invention is a child between the ages of seven and twelve years old. In particular embodiments, the administration of DHA to children between the ages of one and twelve years of age is effective in inducing the expression of pulmonary surfactant protein-B. In other embodiments, the administration of DHA and ARA to children between the ages of one and twelve years of age is effective in inducing the expression of pulmonary surfactant protein-B.

In the present invention, DHA or ARA, alone or in combination with one another, supplementation is effective in inducing the expression of pulmonary surfactant protein-B, thereby treating or preventing infant or neonatal respiratory distress syndrome, acute respiratory distress syndrome, hyaline membrane disease, pulmonary hypoplasia, autosomal recessive lung disorder, primary pulmonary hypertension, meconium aspiration syndrome, congenital alveolar proteinosis, or any other disease or disorder known to be caused by or linked to a pulmonary surfactant protein-B deficiency.

In the present invention, DHA or ARA, alone or in combination with one another, supplementation is effective in inducing the expression of pulmonary surfactant protein-B for subjects that do not naturally produce enough pulmonary surfactant protein-B. The present invention is also effective in producing pulmonary surfactant protein-B for subjects that have a gene mutation that does not allow the natural pulmonary surfactant protein-B that they produce to effectively reduce the surface tension in the alveoli.

The present invention is also beneficial in that it helps provide normal lung development, decreases the incidence of inflammation and infection, increases the lung capacity, stabilizes the fluid system in the lungs, and protects against edema in infants.

In certain embodiments of the invention, DHA or ARA, alone or in combination with one another, are effective in inducing the expression of pulmonary surfactant protein-B in an animal subject. The animal subject may be one that is in need of elevated levels of pulmonary surfactant protein-B. The animal subject is typically a mammal, which can be domestic, farm, zoo, sports, or pet animals, such as dogs, horses, cats, cattle, and the like.

The present invention is also directed to the use of DHA or ARA, alone or in combination with one another, for the preparation of a composition or medicament for inducing the expression of pulmonary surfactant protein-B. In this embodiment, the DHA or ARA, alone or in combination with one another, may be used to prepare a composition or medicament for the elevation of pulmonary surfactant protein-B levels in any human or animal neonate. For example, the composition or medicament could be used to elevate the levels of pulmonary surfactant protein-B in domestic, farm, zoo, sports, or pet animals, such as dogs, horses, cats, cattle, and the like. In some embodiments, the animal is in need of elevation of pulmonary surfactant protein-B levels.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLE 1

This example illustrates the influence of zero, moderate, and high levels of DHA on the induction of pulmonary surfactant protein-B expression in term baboons from 2 to 12 weeks of age.

### Methods

### Animals

All animal work took place at the Southwest Foundation for Biomedical Research (SFBR) located in San Antonio, Tex. Animal protocols were approved by the SFBR and Cornell University Institutional Animal Care and Use Committee (IACUC). Animal characteristics are summarized in Table 1.

TABLE 1
Baboon Neonate Characteristics

| | |
|---|---|
| Number of animals | 14 |
| Gender | 10 female, 4 male |
| Conceptional age at delivery (days) | 181.8 ± 6.2 |
| Birth weight (g) | 860.3 ± 150.8 |
| Weight at 12 weeks (g) | 1519.1 ± 280.7 |
| Weight gain (g) | 658.8 ± 190.4 |

Fourteen pregnant baboons delivered spontaneously around 182 days gestation. Neonates were transferred to the nursery within 24 hours of birth and randomized to one of three diet groups. Animals were housed in enclosed incubators until 2 weeks of age and then moved to individual stainless steel cages in a controlled access nursery. Room temperatures were maintained at temperatures between 76° F. to 82° F., with a 12 hour light/dark cycle. They were fed on experimental formulas until 12 weeks of life.

### Diets

Animals were assigned to one of the three experimental formulas, with LCPUFA concentrations presented in Table 2.

TABLE 2
Formula LCPUFA composition

| | C | L | L3 |
|---|---|---|---|
| DHA (%, w/w) | 0 | 0.42 ± 0.02 | 1.13 ± 0.04 |
| DHA | 0 | 21.3 ± 1.0 | 62.8 ± 1.9 |
| (mg/100 kcal) | | | |
| ARA (%, w/w) | 0 | 0.77 ± 0.02 | 0.71 ± 0.01 |
| DHA | 0 | 39.4 ± 0.9 | 39.2 ± 0.7 |
| (mg/100 kcal) | | | |

Target concentrations were set as shown in brackets and diets were formulated with excess to account for analytical and manufacturing variability and/or possible losses during storage. Control (C) and L, moderate DHA formula, are the commercially available human infant formulas Enfamil® and Enfamil LIPIL®, respectively. Formula L3 had an equivalent concentration of ARA and was targeted at three-fold the concentration of DHA.

Formulas were provided by Mead Johnson & Company (Evansville, Ind.) in ready-to-feed form. Each diet was sealed in cans assigned two different color-codes to mask investigators. Animals were offered 1 ounce of formula four times daily at 07:00, 10:00, 13:00 and 16:00 with an additional feed during the first 2 nights. On day 3 and beyond, neonates were offered 4 ounces total; when they consumed the entire amount, the amount offered was increased in daily 2 ounce increments. Neonates were hand fed for the first 7-10 days until independent feeding was established.

### Growth

Neonatal growth was assessed using body weight measurements, recorded two or three times weekly. Head circumference and crown-rump length data were obtained weekly for each animal. Organ weights were recorded at necropsy at 12 weeks.

### Sampling and Array Hybridization

Twelve week old baboon neonates were anesthetized and euthanized at 84.4±1.1 days. RNA from the precentral gyrus of the cerebral cortex was placed in RNALater according to vendor instructions and was used for the microarray analysis and validation of microarray results.

Microarray studies utilizing baboon samples with human oligonucleotide arrays have been successfully carried out previously. Cerebral cortex global messenger RNA in the three groups was analyzed using Affymetrix Genechip™ HG-U133 Plus 2.0 arrays. See http://www.affymetrix.com/products/arrays/specific/hgu133plus.affx. The HG-U133 Plus 2.0 has >54,000 probe sets representing 47,000 transcripts and variants, including 38,500 well-characterized human genes. One hybridization was performed for each animal (12 chips total). RNA preparations and array hybridizations were processed at Genome Explorations, Memphis, Tenn. <http://www.genome-explorations.com>. The completed raw data sets were downloaded from the Genome Explorations secure ftp servers.

### Microarray Data Analysis

Raw data (.CEL files) were uploaded into Iobion's Gene Traffic MULTI 3.2 (Iobion Informatics, La Jolla, Calif., USA) and analyzed by using the robust multi-array analysis (RMA) method. In general, RMA performs three operations specific to Affymetrix GeneChip arrays: global background normalization, normalization across all of the selected hybridizations, and log2 transformation of “perfect match” oligonucleotide probe values [42]. Statistical analysis using the significance analysis tool set in Gene Traffic was utilized to perform Multiclass ANOVA on all probe level normalized data. Pairwise comparisons were made between C vs L and C vs L3 and all probe set comparisons reaching P<0.05 were included in the analysis. Gene lists of differentially expressed probe sets were generated from this output for functional analysis.

### Measurement and Analysis of Data:

The primary parameter evaluated was regulation of global gene expression using Oligonucleotide Affymetrix DNA microarrays. Data were expressed as mean ±SD. Changes in gene expression were evaluated using a random coefficient regression model to detect effects of DHA and ARA supplementation.

For every parameter, a slope and intercept was determined for each subject. Diet treatment was the fixed effect and random effects included subject, age, and the age * diet interaction. Regression analysis calculated intercepts using postnatal age—2 weeks, the initial sampling time point. Using an analysis of covariance, slopes were compared between diet groups with the baseline C group as the covariate. Anthropometric measurements were also assessed using a regression model to examine systematic effects of diet over time. Statistical analyses were performed using SAS for Windows 9.1 (SAS Institute, Cary, N.C.), with significance declared at p<0.05.

Tissue was collected from the baboon liver, thymus, spleen, ileum, colon, skeletal muscle, heart, lung, kidney, pancreas, ovary/testis, skin and fur, adipose, and spinal cord. Oligonucleotide Affymetrix DNA microarrays (available from http://www.affymetrix.com) were used to determine the changes in global gene expression influenced by varying amounts of DHA and ARA.

### Results

Growth outcomes were assessed using animal body weight, head circumference and crown-rump length. Statistical analyses revealed no significant differences among diet treatments (p>0.37). Anthropometric measurements indicated normal neonatal growth and physical development.

The results of the Oligonucleotide Affymetrix DNA microarray showed that the administration of 0.33% DHA and 0.67% ARA, as a percentage of total fatty acids, induces the expression of pulmonary surfactant protein-B by 3% when compared to an unsupplemented group. The administration of 1.00% DHA and 0.67% ARA, however, induces the expression of pulmonary surfactant protein-B by 35% when compared to an unsupplemented group. Therefore, it is clear that supplementation of 1.00% DHA and 0.67% ARA can unexpectedly and significantly induce the expression of pulmonary surfactant protein-B.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part. For example, while methods for the production of a commercially sterile liquid nutritional supplement made according to those methods have been exemplified, other uses are contemplated. Therefore, the spirit and scope of the appended claims should not be limited to the description of the preferred versions contained therein.

What is claimed is:

1. A method for inducing the expression of pulmonary surfactant protein-B in an infant, the method comprising administering to the infant a therapeutically effective amount of DHA and ARA.

2. The method according to claim 1, wherein the infant is in need of such induced expression of pulmonary surfactant protein B.

3. The method according to claim 1, wherein the infant is at risk for developing RDS.

4. The method according to claim 1, wherein the increased expression of pulmonary surfactant protein-B in an infant treats or prevents a disorder selected from the group consisting of neonatal respiratory distress syndrome, acute respiratory distress syndrome, hyaline membrane disease, pulmonary hypoplasia, autosomal recessive lung disorder, primary pulmonary hypertension, meconium aspiration syndrome, and congenital alveolar proteinosis.

5. The method according to claim 1, wherein the therapeutically effective amount of DHA is between about 15 mg per kg of body weight per day and 60 mg per kg of body weight per day.

6. The method according to claim 1, wherein the therapeutically effective amount of ARA is between about 20 mg per kg of body weight per day and 60 mg per kg of body weight per day.

7. The method according to claim 1, wherein the ratio of ARA:DHA by weight is from about 1:3 to about 9:1.

8. The method according to claim 1, wherein the ratio of ARA:DHA by weight is about 2:1.

9. The method according to claim 1, wherein the ratio of ARA:DHA by weight is about 1:1.5.

10. The method according to claim 1, wherein DHA comprises between about 0.33% and 1.00% of fatty acids by weight.

11. The method according to claim 1, wherein the DHA and ARA are administered to the infant during the time period from birth until the infant is about one year of age.

12. The method according to claim 1, wherein the DHA and ARA are administered to the infant in an infant formula.

13. A method for inducing the expression of pulmonary surfactant protein-B in an infant, the method comprising administering to the infant a therapeutically effective amount of ARA and DHA, wherein the ratio of ARA:DHA by weight is about 1:1.5.

14. A method for inducing the expression of pulmonary surfactant protein-B in an infant, the method comprising administering to the infant a therapeutically effective amount of ARA and DHA, wherein the therapeutically effective amount of ARA is between about 20 mg per kg of body weight per day and 60 mg per kg of body weight per day and wherein the therapeutically effective amount of DHA is between about 15 mg per kg of body weight per day and 60 mg per kg of body weight per day.

15. A method for inducing the expression of pulmonary surfactant protein-B in an infant, the method comprising administering to the infant a therapeutically effective amount of DHA, wherein DHA comprises between about 0.33% and 1.00% of fafty acids by weight.

16. A method for inducing the expression of pulmonary surfactant protein-B in an infant, the method comprising administering to the infant DHA.

17. A method for inducing the expression of pulmonary surfactant protein-B in an infant, the method comprising administering to the infant ARA.

18. A method for inducing the expression of pulmonary surfactant protein-B in a child, the method comprising administering to the child DHA.

19. The method according to claim 26, wherein the child is between the ages of one and six years of age.

20. The method according to claim 26, wherein the child is between the ages of about seven and twelve years of age.

21. The method according to claim 26 additionally comprising administering ARA to the child.

22. A method for inducing the expression of pulmonary surfactant protein-B in a child, the method comprising administering to the child ARA.

23. A method for inducing the expression of pulmonary surfactant protein-B in an infant, the method comprising prenatal administration of DHA and ARA to the infant's biological mother.
